(19) United States
(12) Patent Application Publication
(10) Pub. No.: US 2010/0147293 A1
(43) Pub. Date: Jun. 17, 2010

(54) AEROSOL ACTUATOR

(75) Inventors: Alan Langford, Herts (GB); Fergal Hennessy, Co. Clare (IE); Michael Holroyd, Cambridge (GB)

Correspondence Address: RATNERPRESTIA, P.O. BOX 980, VALLEY FORGE, PA, 19482, US

(73) Assignee: Norton Healthcare, Ltd., London (GB)

(21) Appl. No.: 12/713,761

(22) Filed: Feb. 26, 2010

Related U.S. Application Data
(63) Continuation of application No. 10/481,961, filed on Mar. 12, 2007.
(60) Provisional application No. 60/301,296, filed on Jun. 26, 2001.

(51) Int. Cl. A61M 11/00

(52) U.S. Cl. 128/200.23

(57) ABSTRACT

A metered dose aerosol actuator, for use in association with a pressurized medicament container provided with a dispensing metering valve having a valve stem, including a housing adapted to receive the container, a stem block extending upwards from the base of the housing and adapted to engage the valve stem by means of a cavity provided in the stem block, the stem block being provided with a spray orifice adapted to be in flow connection with the valve stem and the cavity, a sump extending downwards from the cavity relative to the spray orifice, wherein the sump has a smooth, rounded interior surface.

### CROSS-REFERENCE TO RELATED APPLICATIONS

This application is a continuation of U.S. patent application Ser. No. 10/481,961, filed Dec. 22, 2003, which is a U.S. national phase application of International Patent Application No. PCT/US2002/20136, filed Jun. 26, 2002, which claims priority to U.S. Provisional Patent Application No. 60/301,296, filed Jun. 26, 2001. The disclosures of U.S. patent application Ser. No. 10/481,961; International Patent Application No. PCT/US2002/20136; and U.S. Provisional Patent Application No. 60/301,296 are each incorporated herein by reference in their entirety for all purposes.

### FIELD OF THE INVENTION

The present invention relates to the field of inhalers used for the delivery of medicaments indicated for the treatment, or alleviation of the effects of any ailment including respiratory complaints and various systemic diseases, via the delivery of a medicament by the pulmonary route.

### BACKGROUND OF THE INVENTION

Current conventional actuators used for the delivery of medicaments include a sump, or well, located in a valve stem block adjacent to the actuator outlet. The shape of the sump is dictated primarily by engineering requirements such as the strength of small molding pins. Current sumps have angles and comers where the medicament being delivered can accumulate and/or deposit, giving rise to a reduction in the dose available and an increase in the occurrence of the blockage of the spray orifice.

### SUMMARY OF THE INVENTION

Thus, there remains a need for improved, superior sumps to address and solve the problem of medicament accumulation and/or deposition and the concomitant reduction in the dose available resulting in variations and inconsistencies of the actual dose administered, blockage of the spray orifice, and medicament flaking.

Disclosed herein is a sump configuration, for use in an actuator for application with a pressurized aerosol container. In particular, the invention relates to the improvement and optimization of drug delivery performance compared with conventional actuators incorporating angular sumps.

The improved sumps, according to aspects of the invention, may be incorporated in a wide range of aerosol actuators. The term “actuator” as used in the present invention includes all types of actuators presently available including but not limited to standard metered dose inhalers or breath operated inhalers. Breath-actuated devices are also known, and have been the subject of many patent applications. For example, GB 1288971; GB 1297993; GB 1335378; GB 1383761; GB 1392192; GB 1413285; W085/01880; GB 2204799; U.S. Pat. No. 4,803,978 and EP 0186280A, which are all incorporated by reference, describe inhalation-actuated dispensing devices for use with a pressurized aerosol dispensing container.

A typical breath activated or operated device includes a dispensing container comprising a valve capable of releasing a metered amount of the aerosol contents, when an internal spring operating the valve is compressed by a sufficient amount. The dispensing device often comprises a chamber having a mouthpiece, air inlets, actuating means for causing the actuation of the valve in the dispensing container, a latching means for releasably retaining said metering valve in a charged position, and an inhalation responsive means for releasing the latch, such that a metered amount of aerosol compound is discharged into the region of the mouthpiece. The overall objective is to give co-ordination of discharge of medicament from the aerosol container with inhalation of the patient, thus allowing the required or intended dose of medicament to reach the bronchial passages of the lungs, or other target sites in the pulmonary system reliably.

The present invention works in conjunction with the medicament container, generally in the form of a cylindrical canister which, when activated, engages the valve stem against the rounded sump and discharges a metered dose of formulation. The rounded sump allows for smooth delivery of a greater percentage of the metered dose of the formulation as delivered by the metering valve of the canister towards the spray orifice and reduces the amount of measured dose lost in the delivery process by deposition and/or accumulation in and around the angles and corners of current sumps.

According to aspects of the invention disclosed herein is a metered dose aerosol actuator, for use in association with a pressurized medicament container provided with a dispensing metering valve having a valve stem, comprising a housing adapted to receive the container, a stem block, extending upwards from the base of the housing and adapted to engage the valve stem by means of a cavity provided in the stem block, the stem block being provided with a spray orifice adapted to be in flow connection with the valve stem and the cavity, a sump extending downwards from the cavity, relative to the spray orifice, wherein the sump has a smooth, rounded interior surface.

The improved, rounded sump provides reduced deposition of the medicament by the provision of a continuous flow path for the formulation to be directed towards the spray orifice.

The improved sump is dimensioned and configured to include smooth surfaces which reduce the deposition of medicament accumulation and therefore improves product delivery through the spray channel to the spray orifice.

Preferably, the actuator according to aspects of the invention, including the improved sump should be used for formulations including ingredients such as excipients, surfactants or solvents, such as ethanol, glycerols, glycols and generally polyols, which are likely to promote deposition and/or accumulation.

The improved sump can be incorporated equally well in a wide range of inhalers such as a breath operated inhaler (BOI) and a metered dose inhaler (MI).

With respect to current inhaler devices, the smooth, rounded sump is molded in a continuous form to reduce the deposition of formulation around the sump area and improve the actuator performance, with the advantage that it contains no angles or corners where the medicament being delivered could accumulate and/or deposit, giving rise to a reduction in the dose available and blockage of the spray orifice

The smooth, rounded sump gives rise to an increase in the actual metered dose available at the spray orifice, via the spray channel, as a result of the occurrence for the blockage of said spray orifice.

The actuator of the invention, with the improved sump is particularly useful for the administration of medicinal pressurized aerosols, including but not limited to those containing HFA propellants and more particularly those containing suspension formulations. The use of the improved sump can be considered for any actuator in which it would improve and/or optimize the actuator performance under laboratory conditions and under the conditions of or mimicking real life usage.

Preferably, the sump is U-shaped.

The actuator may be manufactured from conventional materials used for actuators (i.e., from a plastics material, such as polypropylene, acetal or molded polystyrene). It may, however, be manufactured from metal or another suitable material.

The invention may be embodied in other specific forms without departing from the spirit or essential characteristics thereof. The present embodiments are therefore to be considered in all respects illustrative and not restrictive.

### BRIEF DESCRIPTION OF THE DRAWINGS

A preferred embodiment of the actuator, according to aspects of the invention, will now by described, by way of example, with reference to the accompanying drawings, in which:

FIG. 1 is a side view in partial section of an aerosol actuator according to aspects of the invention fitted with a pressurized medicament container;

FIG. 2 is an expanded view of the stem-block of the actuator shown in FIG. 1, in horizontal section, omitting the valve stem; and

FIG. 3 is a vertical section of the stem-block shown in FIG. 2, along the line I-I.

### DETAILED DESCRIPTION OF THE INVENTION

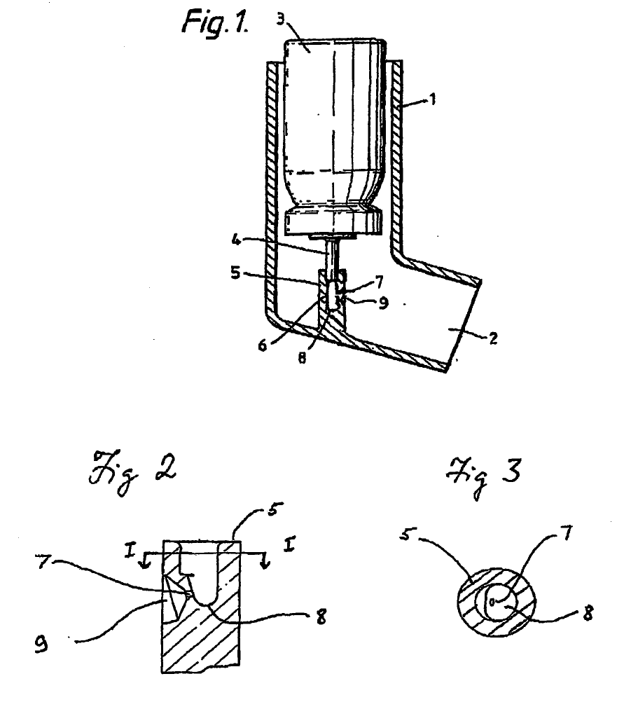

Referring first to FIG. 1, an aerosol actuator comprises a generally cylindrical housing 1 having a mouthpiece 2. The housing 1 receives a container 3 of pressurized medicament, the container being provided at one end with a metering valve including a valve stem 4. The valve stem 4 is seated in the stem block 5. The stem block includes an internal cavity 6 provided with an outlet channel 7 which terminates on the external surface of the stem block 5, with a spray orifice 9, which is directed towards the mouthpiece 2. The valve stem 4, internal cavity 6, outlet channel 7 and spray orifice 9 form a continuous flow path.

As can be seen more clearly form FIG. 2 and FIG. 3, the internal cavity 6 tapers downwards towards the base of housing 1, below the outlet channel 7, terminating in a smooth, rounded, U-shaped sump 8.

In use, a patient inhales at the mouthpiece 2 while simultaneously urging the medicament container 3 towards the stem block 5. The relative movement of the container 3 and the valve stem 4 causes the metering valve to discharge, dispensing a metered dose of pressurized formulation into the valve stem 4. The medicament then passes through the internal cavity 6 of the stem block 5, the outlet channel 7, and the spray orifice 9, after which medicament in aerosol form is inhaled by the patient through the mouthpiece 2.

The sump 8 is molded in a continuous form which gives the advantage that it contains no angles or corners where the medicament being delivered could accumulate and/or deposit, giving rise to a reduction in the dose available and blockage of the spray orifice 9 and thus improving the quantity and quality of the metered dose delivered from the container 3.

Medicament is not accumulated or deposited in the sump 8 due to the smooth rounded effect of the sump 8 which acts with the continuous flow path defined by the valve stem 4, internal cavity 6, spray channel 7 and spray orifice 9 to prevent blockage of the spray channel 7 or spray orifice 9 and improves product delivery through the spray orifice 9.

What is claimed:

1. A metered dose aerosol actuator for use in dispensing medicament from a pressurized medicament container having a valve stem at one end for dispensing the medicament and a closed end opposite the valve stem, the actuator comprising:
a housing adapted to receive the container in an assembled position, the housing having a base and an open end opposite the base, the container in the assembled position being oriented with the valve stem proximate to the base and the closed end proximate to the open end so that when the housing is oriented in a use position with the open end facing upwardly, the container will be in an inverted orientation with the valve stem facing downwardly and the closed end facing upwardly;
a stem block extending from the base of the housing toward the open end of the housing;
a cavity in the stem block defining a mating surface that is adapted to engage the valve stem of the medicament container with the container in the assembled position;
a spray orifice in the stem block in fluid communication with the cavity via an outlet channel; and
a sump in the cavity extending from the outlet channel toward the base of the housing, the sump being molded in a continuous form and having a continuous smooth, rounded interior surface without angles or corners where the medicament being dispensed could accumulate or deposit.

2. An actuator according to claim 1, wherein the sump is “U” shaped.

3. An actuator according to claim 1, wherein the stem block is mounted on the base of the housing in a fixed position relative to the housing.

4. An actuator according to claim 1 further comprising a tapered surface that extends between the mating surface of the cavity and the outlet channel, wherein the mating surface is substantially flat and planar.

5. An actuator according to claim 4, wherein the tapered surface forms an acute angle with respect to a longitudinal axis of the stem block.

6. An actuator according to claim 1, wherein the actuator is composed of a plastic material.

7. A metered dose aerosol actuator for use in dispensing medicament from a pressurized medicament container having a valve stem at one end for dispensing the medicament and a closed end opposite the valve stem, the actuator comprising:
a housing adapted to receive the container in an assembled position, the housing having a base, the container in the assembled position being oriented with the valve stem proximate to the base so that when the housing is oriented in a use position with the base facing downwardly, the container will be in an inverted orientation with the valve stem facing downwardly and the closed end facing upwardly;
a stem block mounted on the base of the housing in a fixed position relative to the housing;
a cavity in the stem block defining a mating surface that is adapted to engage the valve stem of the medicament container with the container in the assembled position;
a spray orifice in the stem block in fluid communication with the cavity via an outlet channel; and
a sump in the cavity extending from the outlet channel toward the base of the housing, the sump being molded in a continuous form and having a continuous smooth, rounded interior surface without angles or corners where the medicament being dispensed could accumulate or deposit.

8. An actuator according to claim 7, wherein the sump is “U” shaped.

9. An actuator according to claim 7 further comprising a tapered surface that extends between the mating surface of the cavity and the outlet channel, wherein the mating surface is substantially flat and planar.

10. An actuator according to claim 9, wherein the tapered surface forms an acute angle with respect to a longitudinal axis of the stem block.

11. An actuator according to claim 7, wherein the actuator is composed of a plastic material.
